(19) United States
(12) Patent Application Publication
(10) Pub. No.: US 2023/0012087 A1
(43) Pub. Date: Jan. 12, 2023

(54) SKIN MOISTURIZING COMPOSITIONS

(71) Applicant: OCuSOFT, Inc., Rosenberg, TX (US)

(72) Inventors: Paramita Sarkar, Richmond, TX (US); Troy Smith, Richmond, TX (US)

(21) Appl. No.: 17/861,588

(22) Filed: Jul. 11, 2022

Related U.S. Application Data
(60) Provisional application No. 63/220,888, filed on Jul. 12, 2021.

(51) Int. Cl. A61K 8/55; A61K 8/34; A61K 8/73; A61K 8/67; A61K 8/04; A61K 8/02; A61K 8/14

(52) U.S. Cl. CPC A61K 8/553; A61K 8/345; A61K 8/735; A61K 8/671; A61K 8/678; A61K 8/676; A61K 8/042; A61K 8/0208; A61K 8/14

(57) ABSTRACT

A topical skin moisturizing composition comprising: (a) one or more phospholipids; (b) a plurality of demulcents; (c) one or more polymeric moisturizing agents; (d) one or more preservatives; (e) one or more vitamins; and (f) an aqueous vehicle. The moisturizing composition can have an osmolality in the range of 80 mOsm/kg to 500 mOsm/kg. The composition is configured for use on the face and, particularly for application on and around the delicate eye area including the eyelids.

### CROSS-REFERENCE TO RELATED APPLICATION

This application claims the benefit of Provisional U.S. Patent Application No. No. 63/220888, filed Jul. 12, 2021, entitled “SKIN MOISTURIZING COMPOSITIONS”, the entire content and disclosure of which, both express and implied, is incorporated herein by reference.

### FIELD OF THE INVENTION

The present invention relates generally to moisturizing compositions for the skin, specifically for the face, eyelids and ocular region, and to methods of preparing and using such compositions.

### BACKROUND

The integumentary system is the set of organs forming the outermost layer of a human's body. The integumentary system comprises the skin and its appendages. The skin acts as a physical barrier between the external environment and the internal environment that it serves to protect and maintain. The skin also serves to maintain water balance, protect deeper tissues, regulate body temperature, and detect pain, sensation, pressure, and temperature. In general, skin helps maintain a body's equilibrium.

The skin around the eyes/ocular region is unique, because it is thinner than the rest of the skin on the body and lacks the fat which cushions the rest of the body's skin. It is unable to readily retain moisture and is highly susceptible to dryness. Dryness of the skin, left untreated, may lead to discomfort, irritation, inflammation, and itching. Dryness of the skin can also affect elasticity, which can lead to premature aging and wrinkles.

Additionally, the ocular region is highly vulnerable to irritants. These irritants include but are not limited to environmental imbalances and physiological conditions. Such irritants can exacerbate discomfort and irritation caused by skin dryness in the ocular region.

Dry and irritated skin is treated with moisturizers. Moisturizers can reduce the chance of developing extreme dryness and can combat skin discomfort and irritation. Often, moisturizers contain ingredients and chemicals which are unsuitable for application in the ocular region. For example, hydrocortisone cream can be used to treat irritation of the skin, including on the face. However, hydrocortisone cream is generally too harsh to be used on the eyelid or in the ocular region. Thus, the sensitivity can make it difficult to find a quick-acting, soothing delivery of key components to relieve dry skin, discomfort, and irritation while also being mild enough for ocular region application.

### SUMMARY OF THE INVENTION

According to an embodiment, a skin moisturizing composition includes: phosphatidylcholine; a mixture of propylene glycol and glycerol; a polymeric moisturizer; and purified water. The composition can be encapsulated in a plurality of liposomes. The polymeric moisturizer consists essentially of sodium hyaluronate. The composition further comprises a mixture of vitamins, the vitamins selected from the group consisting of Vitamin A and its derivatives, Vitamin C and its derivatives, Vitamin E and derivatives and mixtures thereof. The composition further comprises 0.001 to 5.0% w/w of a preservative, such as, 2-Phenoxyethanol. The composition further comprises a nonionic surfactant, such as, Polysorbate 20. The composition is configured as non-irritant to the skin and non-toxic to eye tissue.

According to an embodiment, the skin moisturizing composition includes: 0.01% to 10% w/w of phosphatidylcholine; 0% to 5% w/w of propylene glycol and 0% to 5% w/w of glycerol; 0.005% to 10% w/w of the polymeric moisturizer; 0.001% to 1.5% w/w of Vitamin A, 0.001% to 1.5% w/w of Vitamin C and 0.005% to 1.5% w/w of Vitamin E; 0.05% to 10% w/w of Polysorbate 20 and 60%-98% w/w purified water. The composition has an osmolality in the range of 80 mOsm/kg to 500 mOsm/kg and a pH in the range of 4.0 to 7.0.

The composition is in a liquid, spray, mist, gel, cream or lotion form. In one or more embodiments, the composition is a liposomal spray, wherein the liposomal spray includes a liposomal carrier encapsulating the composition. Alternately, the composition can be dispersed with liposomes.

In another embodiment, a skin moisturizing kit comprises: a spray bottle, wherein the spray bottle includes a composition including phosphatidylcholine; a mixture of propylene glycol and glycerol; a polymeric moisturizer; and purified water. The spray bottle dispenses the composition as a liposomal spray. In one or more embodiments, a dose equal to or less than 160 μL is dispensed per spray.

According to another embodiment, a method for moisturizing the skin comprises: providing a composition including phosphatidylcholine; a mixture of propylene glycol and glycerol; a polymeric moisturizer; and purified water. The composition is encapsulated in or dispersed with a liposome carrier. A desired amount of the composition can be topically applied to skin in need thereof.

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the skin care composition, unless otherwise specified. Additionally, all composition percentages are based on totals equal to 100 wt. %, unless otherwise specified.

The disclosed compositions and methods for their use can “comprise,” “consist essentially of,” or “consist of ” any of the ingredients or steps disclosed throughout the specification. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claims, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. In one non-limiting embodiment, the terms are defined to be within 5%. The term “substantially” and its variations are defined as being largely but not necessarily wholly what is specified as understood by one of ordinary skill in the art, and in one non-limiting embodiment substantially refers to ranges within 0.01% to 5%.

As used herein, the term “effective amount” of a composition refers to an amount sufficient to elicit the desired biological response. As will be appreciated by those of ordinary skill in this art, and effective amount of a substance may vary depending on such factors as the desired biological endpoint, the patient, etc. The terms effective amount and clinically effect may be used interchangeably herein. For example, an effective amount/clinically effective amount of the composition is the amount that can be used to facilitate skin moisturization and treat irritation and discomfort of the skin.

While the specification concludes with the claims particularly pointing and distinctly claiming the invention, it is believed that the present invention will be better understood from the following description

The highly sensitive nature of the ocular region, which is even greater than facial skin in general, is a major concern in the formulation of any composition to be used around the eyes. According to an embodiment, a moisturizing composition (“composition”) is configured to treat dryness, irritation, and discomfort of the skin around the eyes (ocular region) while minimizing the exacerbating effects of environmental imbalances and physiological conditions. The composition contains an aqueous vehicle having at least one phospholipid and one or more vitamins as active pharmaceutical ingredients (APIs). The composition further includes a mixture of at least two demulcents (also referred to as emollients when used topically), one or more polymeric moisturizing agents, preservatives and surfactants. The composition is typically delivered by encapsulation in/dispersed with a liposomal carrier. A liposome is a spherical vesicle having at least one lipid bilayer. Liposomes are specialized delivery vehicles that enhance the capabilities of the APIs.

Phospholipids are used in the composition as a dispersant and to provide a barrier for the skin to seal in any active ingredients aimed at treating dry, irritated skin. Phospholipids are a type of lipid or fat molecule that are described as having hydrophilic heads and hydrophobic tails. This dual nature means that a phospholipid attracts water and absorbs it, which is a crucial component in keeping skin hydrated. Phospholipids provide a barrier for the skin and work much like a second skin by mimicking the skin's natural lipids to seal in any product added to the skin. In this way, phospholipids can help hydrating ingredients penetrate the skin and can ensure active ingredients are delivered deep into the skin. Moreover, phospholipids can help retain moisture thereby reducing skin dryness. Phospholipids are made up of two fatty acids, a phosphate group, and a glycerol molecule. Phospholipids are complex lipids, as distinguished from the simple lipids and other fat-soluble cell components.

The composition has at least one phospholipid. In one or more embodiments, the phospholipid is phosphatidylcholine. However, the phospholipid can be selected without limitation, from the group consisting of phosphatidylcholine, phosphatidic acid, phosphatidyl ethanolamine, phosphatidylinositol, phosphatidyl glycerol, phosphatidyl serine, ceramides, sphingomyelin, sphingolipids, and derivatives and mixtures thereof. A total concentration of the at least one phospholipid in the composition is in the range of 0.01% to 10% w/w. In one or more embodiments, the total concentration of phosphatidylcholine in the composition is in the range of 0.1% to 5% w/w.

The composition further includes one or more vitamins. Suitable vitamins include Vitamin A and its derivatives, Vitamin B complexes, Vitamin C and its derivatives, and Vitamin E and its derivatives. These can be used independently or in combination with others in the composition.

Vitamin A is a group of unsaturated nutritional organic compounds that includes retinol, retinyl palmitate, and several provitamin A carotenoids, such as beta-carotene. In the skin, retinol is converted to retinaldehyde and then to retinoic acid, which modulates cellular processes in both the epidermis and dermis layers of the skin. Vitamin A stimulates production of new skin cells. In the absence of Vitamin A, the skin can become overly dry. In addition to helping combat dryness, Vitamin A can also be used to treat acne and fine lines. The term “Vitamin A and its derivatives” further comprises all-trans retinoic acid, also known as tretinoin, and its isomers, such as 13-cis retinoic acid, isotretinoin, and their respective derivatives. In an embodiment, the total concentration of Vitamin A and its derivatives in the composition is in the range of 0.001% to 1.5% w/w. In one or more embodiments, the composition includes Vitamin A in the range of 0.005% to 0.5% w/w.

Vitamin B complexes are a class of water-soluble vitamins that includes thiamine, panthenol, folic acid, cyanocobalamin, methycobalamin, niacin and biotin. Vitamin B complexes can be used to moisturize dry skin. In addition to helping combat dryness, Vitamin B complexes can also be used to treat fine lines and dullness.

Vitamin C is a natural water-soluble vitamin, an essential nutrient, and an antioxidant that protects against environmental stressors or imbalances by scavenging free radicals in the skin and promotes collages production, which gives skin support and structure. The pure form is L-ascorbic acid. Vitamin C is required for growth and repair of tissue in all parts of the body, including the skin. The term “Vitamin C and its derivatives” further comprises sodium ascorbyl phosphate, ascorbyl palmitate, retinyl ascorbate, tetrahexyldecyl ascorbate, and magnesium ascorbyl phosphate. In an embodiment, the total concentration of Vitamin C and its derivatives in the composition is in the range of 0.001% to 1.5% w/w. In one or more embodiments, the composition includes Vitamin C in the range of 0.005% to 0.5% w/w.

Vitamin E is a group of eight fat soluble compounds that include four tocopherols and four tocotrienols. The pure form is alpha-tocopherol. Vitamin E is an antioxidant, reduces inflammation and helps treat dry skin by promoting cell turnover and regeneration. The term “Vitamin E and its derivatives” further comprises synthetic tocopheryl acetate, tocopheryl linoleate, tocopherol sorbate, and other esters of tocopheryl. In an embodiment, the total concentration of Vitamin E and its derivatives in the composition is in the range of 0.005% to 1.5% w/w. In one or more embodiments, the composition includes Vitamin E in the range of 0.01% to 0.5% w/w.

One or more polymeric moisturizing agents are used in the composition to modulate the moisture content of the skin and protect the skin barrier from drying. Suitable polymeric moisturizing agents can include, without limitations, hyaluronic acid and its derivatives, PEG-400, PEG-600, PEG-1200, etc., tremella fuciformis polysaccharide (TFPS), polysaccharides such as chondroitin-4-sulfate and chondroitin-6-sulfate, and alginic acid and mixtures and derivatives thereof. These can be used independently or in combination with others in the composition. A total concentration of each of the one or more polymeric moisturizing agents in the composition is in the range of 0.005% to 10% w/w.

Hyaluronic acid is a disaccharide polymer molecule which can help bind water to collagen thereby moisturizing the skin. The term “hyaluronic acid and its derivatives” include esterified derivatives and silicon derivatives, single or multiple esters of primary or secondary alcohol group, or acid functional groups of hyaluronic acid, meaning any derivative having a carbon-based chain. Sodium salt of hyaluronic acid—sodium hyaluronate is an exemplary hyaluronic acid derivative used in the composition. In an embodiment, the composition includes 0.01% to 0.5% sodium hyaluronate.

PEGs (polyethylene glycols) are petroleum-based compounds that can be used as emollients, as emulsifiers, and as carriers that can help deliver other ingredients deeper into the skin. Moreover, PEGs have anti-microbial properties. The anti-microbial activity will vary depending on the total concentration, the presence of one or more surfactants, and the pH of the composition. Since the weight and size of the polyethylene glycols is too large to penetrate beyond the surface of the skin, these compounds can be used as emollients to help soften and moisturize the skin. In an embodiment, the composition includes 0% to 5% PEG-400.

Tremella fuciformis polysaccharide is a sugar molecule extracted from the edible fruit bodies of the Silver Ear mushroom in China. Its sugar constituents include glucuronic acid, which is also one of the key components of hyaluronic acid. Like hyaluronic acid, tremella fuciformis polysaccharide helps bind water to collagen thereby moisturizing the skin. In an embodiment, the composition includes 0.01% to 0.5% tremella fuciformis polysaccharide.

Alginic acid, or algin, is a polysaccharide that is widely distributed in the cell walls of brown algae. The term “alginic acid and its derivatives” further comprises sodium alginate, calcium alginate, alginate hydrogels, propylene glycol alginate, and other conjugate bases, salts, and esters. While typically used as a thickener or emulsifier, alginates are also known to have water retention properties.

The composition can further include one or more lubricants and emollients (demulcents) selected from the group consisting of glycerin, propylene glycol, mineral oil, medium chain triglycerides, coconut oil, and derivatives and mixtures thereof. Other emollients can include colloidal oatmeal, urea, sorbitol, nut oils and derivatives, floral extracts such as rose extract or fruit extract, liquid polyols, medium chain diglycerides, and silicone oil.

The emollients typically operate by lubricating the skin, making it difficult for water to evaporate, thereby keeping the skin moist. Consequently, the inclusion of at least one demulcent or emollient in the composition helps the composition make the ocular region softer and more hydrated. In an exemplary embodiment, the composition can include at least two demulcents in the composition. For instance, the composition can include about 0% to about 5% w/w of propylene glycol and about 0% to about 5% w/w of glycerin or glycerol.

Propylene glycol is non-toxic to the eyes and ocular region. Propylene glycol works as a humectant by attracting moisture to the skin, and consequently hydrating and moisturizing it. Propylene glycol is also used to dissolve substances in a product formulation to get them to mix and interact properly. Propylene glycol is used in the composition because it forms an oily layer on the skin and prevents water loss. Furthermore, propylene glycol can be used to reduce the thickness of the composition, which helps it spread across the skin better and improves how well the components of the composition are absorbed. Similarly, glycerol is also known to be non-toxic to the eyes and ocular region. Glycerol acts as an emollient to soften and soothe the skin. In an embodiment, the composition includes 0% to 2.00% propylene glycol and 0% to 2.00% glycerol.

One or more preservatives are also used in the composition. Because of the highly sensitive nature of the ocular region, all preservatives act as an irritant to the eye or ocular region if used in a high enough quantity. One aspect of the ocular moisturizing composition is that it contains a very low concentration of preservatives, preferably less than 1.5% w/w.

Preservatives include, without limitation, phenoxyethanol, hexylene glycol, caprylyl glycol, parabens such as propylparaben and butylparaben, benzoic acid, sodium benzoate, sorbic acid, potassium sorbate, levulinic acid, Leuconostoc/Radish Root ferment filtrate and anisic acid, and mixtures and derivatives thereof.

The composition contains a very low concentration of preservatives that are effective against a wide spectrum of microorganisms. Phenoxyethanol, at low doses, has been shown to be an effective preservative for moisturizers that is effective against a wide spectrum of microorganisms. In an embodiment, a total concentration of phenoxyethanol used in the composition is in the range of 0.05% to 2% w/w. In another embodiment, the composition includes 0.10% to 1.00% 2-Phenoxyethanol. To lower the effective dosage of phenoxyethanol, in some embodiments of the composition, a small dose of Leuconostoc/Radish Root ferment filtrate is added. The Leuconostoc/Radish Root ferment filtrate is a natural preservative that comes from radishes fermented with Leuconostoc kimchii, a lactic acid bacteria that has been used to make the traditional Korean dish, kimchi. During the fermentation process, a peptide is secreted from the bacteria that has significant antimicrobial properties. The Leuconostoc/Radish Root ferment filtrate is water soluble, naturally derived, virtually odourless, relatively heat stable (up to 60° C. or 140° F.), has a broad spectrum of antibacterial activity, and is generally accepted as having a low to no irritation potential in concentrations less than 4% w/w. The benefits of using Leuconostoc/Radish Root ferment filtrate as a preservative in ocular skin moisturizing products are quite numerous. The ingredient is a moisturizer and conditioner by itself, so it not only serves as a non-toxic preservative, but also contributes to the soothing moisturizing effect of the composition.

The aqueous vehicle can be deionized water or purified water USP. The composition can include 60% to 98% water. The composition is physiologically compatible and iso-osmolar. However, in one or more embodiments, other aqueous vehicles, such as, aqueous fruit or floral extracts can be used. The aqueous vehicle includes one or more components for adjusting osmolality and pH, as needed. Osmolality can be adjusted using sugars, glycerin, propylene glycol and similar ingredients. These can be used independently or in combination with others in the composition. The osmolality of the composition can be adjusted from about 80 mOsm/kg to about 500 mOsm/kg.

One or more surfactants may be required for delivering the composition in spray form. The emulsifying property of surfactants would prevent the separation of the composition. Suitable surfactants include non-ionic surfactants but can include, without limitations, polyoxyethylene modified fatty acid esters and ethers, including, Polysorbate 20, Polysorbate 60 and Polysorbate 80, sugar based surfactants, namely, glucosides, and PEGylated castor oil and its derivates. In certain embodiments, the total concentration of the one or more surfactants in the composition is in the range of 0.05% to 10% w/w. In one embodiment, the composition includes 0.1% to 5% Polysorbate 20.

The composition can further include one or more salts. For example, the salts can include, without limitation, sodium chloride, potassium chloride, sodium and/or potassium mono-and di-basic phosphate, calcium chloride, sodium citrate and combinations of the same. Optimally, the pH of the composition is kept between 3.0 and 9.0. Preferably, the pH of the composition is between 4.0 and 7.0.

The composition can further include one or more ingredients selected from the group consisting of a viscosity agent, an antioxidant, a chelating agent, an osmolality adjuster, a buffer, sensory agents, and mixtures thereof. Viscosity agents include, without limitation, carboxymethyl cellulose, hydroxypropyl methylcellulose, and Carbopol®. Antioxidants include, without limitation, polyphenols such as green tea extract and black tea extract, resveratrol, coenzyme Q10, and curcumin. Chelating agents include, without limitation, tetrasodium EDTA and tetrahydroxypropyl ethylenediamine. Buffers include, without limitation, sodium bicarbonate, acetic acid, adipic acid, boric acid, citric acid, glycolic acid, lactic acid, malic acid, uric acid, ammonium hydroxide, calcium hydroxide, magnesium hydroxide, sodium hydroxide, ammonium phosphate, calcium phosphate, potassium phosphate, sodium phosphate, dimethyl MeA, ethanolamine, triethanolamine, and tromethamine. Sensory agents can include, without limitation, peppermint extracts, spearmint extracts or green apple extracts.

The composition can be provided in a liquid or semisolid dosage form that may be dispensed using a spray, mist, liquid, gel, cream, lotion or via a pre-moistened wipe, such as a fabric wipe.

In one or more embodiments, the moisturizing composition is delivered as a ready to use liposomal spray. The liposomal spray is a hydrating and moisturizing spray containing an embodiment of the moisturizing composition disclosed herein encapsulated within or dispersed with microscopic spherical liposomal vesicles. Liposomal vesicles are known in the art.

In an exemplary embodiment, the liposomal vesicles encapsulate a composition having a phospholipid, namely, phosphatidylcholine; vitamins, namely, Vitamin A, Vitamin C, Vitamin E; a mixture of at least two demulcents, namely, propylene glycol and glycerol/glycerin; a polymeric moisturizer including sodium hyaluronate, tremella fuciformis polysaccharide and PEG-400; a preservative, namely, phenoxyethanol; and a surfactant, namely, polysorbate 20 in an aqueous vehicle. In this embodiment, the phospholipid can range from about 0.05% — 10.00%, the vitamins can range from 0.005% — 5.00%, the demulcents can range from 0%-5.00%, the polymeric moisturizer can range from about 0.005%-10.00%, the preservative can range from 0.05%-2.00%, the surfactant can range from 0.05%-10.00% and water/deionized water Q.S. to 100%. Alternately, the composition can be dispersed with the liposomal vesicles.

The inventors believe the use of a mixture of two demulcents, such as, propylene glycol and glycerine, delivered in a liposomal spray is believed to be novel. The two demulcents are safe and effective and, in combination with a polymeric moisturizing agent (e.g., hyaluronic acid), provides better skin coverage and longer moisturization of the skin in the ocular region.

In another embodiment, a kit is provided. For instance, the composition of the present invention can be included in a kit. The kit can include a container, such as, a bottle, such as, a spray bottle, a pressurized container, a tube, or any other suitable dispenser to hold or retain the composition. The containers can have spray, pump, or squeeze mechanisms. In an exemplary embodiment, the kit includes a spray bottle for dispensing a desired or pre-determined amount of the composition as a liposomal spray. In one or more embodiments, the spray bottle dispenses a dose equal to or less than 160 μL per spray. In other embodiments, the container can be squeezed to dispense a desired amount of the composition. In yet another embodiment, a kit containing one or more wipes (such as, fabric wipes) impregnated with the composition is provided. In yet another embodiment, a kit containing one or more wipes and a container including the composition is provided. The composition can be applied to the wipe prior to use.

The kits disclosed herein can include identifying words or symbols on the surface of the container or packaging for the container/wipes. The kit can also include instructions for using the kit components, including use of any other compositions included in the container. The instructions can include an explanation on the application, use or storage of the composition.

In yet another embodiment, a method of treating skin dryness, specifically on the face, eyelids, and around the eye area, involves providing the composition according to the one or more embodiments disclosed herein. A therapeutically effective amount of the composition may be applied topically to intact skin on the face and around the eyes to provide immediate and prolonged soothing action to dry skin. The composition may be administered several times a day as needed by an individual. The composition may also be administered as needed by a patient in hospitals, doctor's offices, and home healthcare providers.

In another embodiment, a method of manufacturing a moisturizing composition involves preparing a mixture of a required amount of polymeric moisturizing agents, such as, sodium hyaluronate, and water. A required amount of sodium hyaluronate and other moisturizing agents including tremella fuciformis polysaccharide and PEG-400 are slowly added one by one to a required amount of water until a homogenous solution is formed. The mixing can continue for at least 10-30 minutes or until all the agents are fully dispersed/dissolved. The mixture may be warmed to a temperature from about 25° C. to 70° C. to aid in the dispersion of the moisturizing agents. A required amount of glycerin and propylene glycol is added to the mixture with stirring. This mixture is combined with a phospholipid dispersion under moderate to vigorous agitation for a predetermined time period. The phospholipid dispersion can include phosphatidylcholine and a mixture of Vitamins A, C and E. Phenoxyethanol and other preservatives are added to the combined mixture to reach the final desired weight. Agitation is continued for another predetermined time period until a homogenous dispersion is formed. This dispersion is titrated with Polysorbate 20 to provide a composition having a desired surface tension.

The composition can be enclosed in suitable dispenser or containers for dispensation. Alternately, the composition can be encapsulated in or dispersed with liposomal carriers and delivered as a liposomal spray. Liposomal sprays can be uniformly dispersed from a spray nozzle. The substantial equivalence of the composition boosts skin coverage and delivery of the phospholipids, demulcents and vitamins to the skin.

An embodiment of the skin moisturizing composition disclosed herein—identified as Compound D—was tested for its chemical safety in the eye and for its irritation and sensitization to the human skin. The chemical safety of the ocular skin moisturizing composition was tested using the EpiOcular™ test method and the skin irritation and sensitization was evaluated using a repeat insult test patch. This test method used a three-dimensional engineered human tissue-like structure. Basically, 50 μL of the composition was applied to the surface of the tissue in an even layer. The composition was then removed, and the tissue was allowed to recover under normal tissue culture conditions. The medium was then exchanged with a tissue culture medium containing yellow tetrazolium dye MTT 3-(4,5-dimethylthiazol-yl)-2,3-diphenyltetrazolium bromide, which was reduced to its insoluble purple formazan compound. This dark purple compound accumulated in live cells but not in dead cells. After the cells were washed, the reduced purple compound was solubilised in isopropanol and quantified by reading the optical density at 570 nm (OD₅₇₀) using a plate reader. If the result was greater than 60% of the OD₅₇₀ of the control, the substance would be considered non-classified; if the OD₅₇₀ was less than or equal to 60% of the OD₅₇₀ of the control, the substance would be classified as an irritant. Several samples of the present moisturizing composition were tested using this method giving an average OD₅₇₀ that was equal to 99.0% of the control and was thus classified as a non-irritant.

The chemical safety of the moisturizing composition was also tested using 23 test samples to determine the skin irritation and sensitization (contact allergy) potential of the composition after repeated application to the skin of human subjects. Seventy-three subjects enrolled in the study and sixty-three of the subjects completed the study. Approximately 0.02-0.05 ml of the composition test material was dispensed on a 7.5 mm paper disc. Patches containing the material were affixed directly to the skin or the intrascapular region of the back. Patches remained in place for 48 hours after the first application. Thereafter, a series of 8-9 consecutive 24 hour exposures were made 3 times/week for 3 consecutive weeks. Each of the test sites, before and after the application of the sample, was evaluated by trained laboratory personnel. On a scale of 0 (no reaction) to 4 (erythema, induration and bullae), none of the 63 subjects that completed the study had any reaction of any kind during the course of the study.

A liposomal spray composition product having phosphatidylcholine, propylene glycol, glycerol, a sodium salt of hyaluronic acid, Vitamin A and its derivatives, Vitamin C and its derivatives, Vitamin E and derivatives, 2-Phenoxyethanol, Polysorbate 20 and purified water was studied for efficacy. Adult subjects with no known skin sensitivities or current skin conditions were enrolled in the study. The subjects were told to apply the product, with eyes closed, by spraying directly onto eyelids. The subjects were advised to use the product as often as needed during the course of an eight-hour day and asked to keep track of how many times they used the spray during the day. They were asked to record their evaluation of the product on a survey questionnaire against a variety of attributes on a scale of 1-5. The study design, questionnaire, patient screening and results compilation were done by a compounding pharmacy in Texas. The results can be summarized as follows. Twenty two subjects participated in the study and twenty one completed the full evaluation. A majority of the respondents (86%) had a positive review of the product. Greater than 90% found the product to be cooling and refreshing and greater than 80% agreed product was soothing and moisturizing.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

What is claimed is:

1. A skin moisturizing composition, comprising: 0.01% to 10% phosphatidylcholine; a mixture of propylene glycol and glycerol; a polymeric moisturizer; and purified water.

2. The composition according to claim 1, comprising 0.1% to 5% w/w of phosphatidylcholine.

3. The composition according to claim 1, comprising 0% to 5% w/w of propylene glycol and 0% to 5% w/w of glycerol.

4. The composition according to claim 1, wherein the polymeric moisturizer consists essentially of sodium hyaluronate, tremella fuciformis polysaccharide and polyethylene glycol (PEG)-400.

5. The composition according to claim 1, comprising 0.005% to 10% w/w of the polymeric moisturizer.

6. The composition according to claim 1, further comprising a mixture of vitamins, the vitamins selected from the group consisting of Vitamin A and its derivatives, Vitamin C and its derivatives, Vitamin E and derivatives and mixtures thereof.

7. The composition according to claim 6, comprising 0.001% to 1.5% w/w of Vitamin A, 0.001% to 1.5% w/w of Vitamin C and 0.005% to 1.5% w/w of Vitamin E.

8. The composition according to claim 1, further comprising 0.05% to 2% w/w of a preservative.

9. The composition according to claim 8, wherein the preservative is phenoxyethanol.

10. The composition according to claim 1, further comprising a nonionic surfactant.

11. The composition according to claim 1, further comprising 0.05% to 10% w/w of Polysorbate 20.

12. The composition according to claim 1, comprising 60%-98% w/w purified water.

13. The composition according to claim 1, wherein the composition is encapsulated in a plurality of liposomes or dispersed with a plurality of liposomes.

14. The composition according to claim 1, wherein the composition has an osmolality in the range of 80 mOsm/kg to 500 mOsm/kg and a pH in the range of 4.0 to 7.0.

15. The composition according to claim 1, wherein the composition is in a liquid, spray, mist, gel, cream or lotion form.

16. The composition according to claim 1, wherein the composition is configured as non-irritant to the skin and non-toxic to eye tissue.

17. A skin moisturizing kit, comprising:
a spray bottle; and
the composition according to claim 1, wherein the spray bottle is configured to retain the composition.

18. The kit according to claim 17, wherein the spray bottle is configured to dispense a dose equal to or less than 160 μL per spray.

19. A skin moisturizing kit, comprising:
a wipe; and
the composition according to claim 1, wherein the wipe is pre-moistened with the composition.

20. A method for moisturizing the skin, comprising:
providing the composition according to claim 1, wherein the composition is encapsulated in/dispersed with a liposome carrier; and
topically applying a desired amount of the composition to skin in need thereof.
